(19) United States
(12) Patent Application Publication
(10) Pub. No.: US 2012/0070423 A1
(43) Pub. Date: Mar. 22, 2012

(54) ORAL COMPOSITION AND METHOD OF FORMING AND USING SAME

(76) Inventor: Puneet Nanda, Cerritos, CA (US)

(21) Appl. No.: 13/094,740

(22) Filed: Apr. 26, 2011

Related U.S. Application Data
(60) Provisional application No. 61/385,115, filed on Sep. 21, 2010.
(60) Provisional application No. 61/385,510, filed on Sep. 22, 2010.
(60) Provisional application No. 61/413,783, filed on Nov. 15, 2010.

(51) Int. Cl. A61K 38/54; A61P 31/00; A61P 1/02; A61P 1/00

(52) U.S. Cl. 424/94.2

(57) ABSTRACT

An oral composition for the treatment of dry mouth symptoms and other conditions, a method of forming the oral composition, and a method of using the oral composition are disclosed. The oral composition includes a plurality of enzymes to prevent formation of and/or facilitate the break up of biofilm in an oral cavity and a metal ion management system to inhibit growth of gram negative bacteria.

### CROSS REFERENCE TO RELATED APPLICATIONS

This application claims the benefit and priority of Provisional Application No. 61/385,115, filed on Sep. 21, 2010, entitled ORAL RINSE AND METHOD OF FORMING AND USING SAME, Provisional Application No. 61/385,510, filed on Sep. 22, 2010, entitled ORAL RINSE AND METHOD OF FORMING AND USING SAME, and Provisional Application No. 61/413,783, filed on Nov. 15, 2010, entitled ORAL RINSE AND METHOD OF FORMING AND USING SAME.

### FIELD OF THE INVENTION

The present invention generally relates to compositions and methods for promoting and maintaining oral health. More particularly, the invention relates to oral compositions and methods that may be used for the treatment or prevention of “dry mouth,” bad breath, tooth and gum disease, and other conditions.

### BACKGROUND OF THE INVENTION

Maintaining oral health is generally important for a variety of reasons. If oral health is not maintained, a variety of conditions, ranging from bad breath to general health problems, may occur. Such conditions include, for example, tooth decay, tooth coloration, gum disease, tooth loss, and even general health problems, such as heart disease, stroke, poorly controlled diabetes and preterm labor.

Healthy saliva in the oral cavity can help maintain oral health. Saliva is a natural bio wash that helps mitigate growth of biofilms (bacterial colonies that may cause bad breath, tooth decay, gum disease and other disorders) in an oral cavity and helps maintain the pH balance in the mouth, nullifying the effects of, for example, food acids created by undesirable bacteria. Saliva also provides compounds, such as calcium and phosphate ions, to enhance enamel on the surface of teeth, and enzymes that selectively inhibit growth of unwanted bacteria and help fight disease.

Unfortunately, some people may suffer from decreased secretion of saliva, known as xerostomia. Reduced saliva production may be caused by, for example, aging, smoking, radiation, certain medications, certain medical conditions (e.g., Sjögren's syndrome and the like), or dehydration. For example, patients undergoing radiotherapy—particularly to the mouth, oropharynx, or neck area, may experience dry mouth that results from damage to the salivary glands. If left untreated, the reduced saliva production may lead to the oral and/or more general health issues noted above.

Similarly, chemotherapy may cause ulcers and/or pain in the mouth—i.e., mucotosis. If left untreated, the ulcers may become infected, which, in turn, may lead to additional health problems.

In addition, dry mouth or reduced saliva production often leaves one feeling thirsty. Persons with xerostomia may therefore drink too much, in an attempt to relieve the dry mouth, which, in turn, causes fluid overload. Fluid overload may be particularly problematic in patients suffering from other medical conditions, such as chronic kidney disease and other diseases. If left untreated in chronic kidney disease patients, fluid overload may cause heart disease and even death due to ventricular dystrophy.

Typical methods for treatment of reduced saliva production often include application of an oral rinse that includes one or more antibacterial and/or antimicrobial agents. Application of relatively large doses of such agents may be undesirable because, in addition to destroying “bad” or undesirable bacteria, the agents may also kill good (e.g., “healthy” or “normal”) bacteria and/or microbial agents within the oral cavity. In general, it may be desirable to maintain a healthy flora (i.e., normal levels of certain bacteria and other microbials), rather than a reduced flora. In addition, other formulations for treatment of dry mouth are said to have a bad taste. Accordingly, improved oral compositions and methods of making and using the compositions are desired.

### SUMMARY

The present invention provides an improved oral composition that can be used for a variety of treatments and/or early-use prevention of various conditions, including treating or preventing dry mouth, by for example, developing and/or maintaining a healthy or normal flora in an oral cavity and reducing an amount of biofilm in the oral cavity, treating or preventing bad breath, treating common colds, and the like. The ways in which the present invention addresses various drawbacks of now-known oral compositions and methods are discussed in greater detail below. However, in general, the present invention provides a composition for selectively attacking and/or mitigating growth of certain bacterial and microbial compounds, while allowing other bacterial and microbial compounds to survive, for facilitating removal of a biofilm in an oral cavity, for moisturizing a portion of an oral cavity, and for increasing possible space for good bacteria to adhere to the mucosal cavities.

In accordance with various exemplary embodiments of the present invention, a composition includes a plurality of enzymes to promote healthy flora in an oral cavity and a metal ion management formulation. In accordance with exemplary aspects of these embodiments, two or more of the plurality of enzymes are selected from the group consisting of: lysozyme, amylase, amyloglucosidase, glucosidase, peptizyme, and papain. In accordance with a particular aspect, the composition includes lysozyme, amylase, amyloglucosidase, glucoxidase, peptizyme, and papain. In accordance with further exemplary aspects of these embodiments, the metal ion management formulation includes one or more compounds that bind with iron and other metal ions to inhibit growth of gram negative bacteria. Exemplary compounds suitable for inhibiting growth of gram negative bacteria include sodium EDTA, phytic acid, lactoferrin, and various combinations thereof. In accordance with further aspects of these embodiments, a composition includes additional actives, such as zinc gluconate and/or calcium lactate. In accordance with yet further aspects, the composition includes a carrier, which may include solvents, sweeteners, preservatives, thickeners and/or emulsifiers, surfactants, flavorings, pH stabilizers, and the like. The composition may be in a variety of configurations, such as an oral rinse, an oral spray, a foaming liquid, liquid drops, or the like.

In accordance with additional embodiments of the invention, a method of using the composition includes the steps of applying a dose (e.g., about one tablespoon) of the composition including a plurality of enzymes selected from the group consisting of: lysozyme, amylase, amyloglucosidase, glucosidase, peptizyme, and papain (e.g., the composition may include lysozyme, amylase, amyloglucosidase, glucoxidase, peptizyme, and papain) and a metal ion management formulation to an oral cavity, maintaining the composition in the oral cavity for an extended period of time (e.g., about 30 seconds to about one minute), spitting out or removing the composition from the oral cavity, and repeating the process 2-3 times per day or as needed. In accordance with various aspects of these embodiments, the method of using the composition includes treatment or prevention of dry mouth, treatment or prevention of bad breath, treatment of a common cold, or the like.

In accordance with yet additional embodiments of the invention, a method of using the composition includes the steps of spaying a dose of the composition including a plurality of enzymes selected from the group consisting of: lysozyme, amylase, amyloglucosidase, glucosidase, peptizyme, and papain (e.g., the composition may include lysozyme, amylase, amyloglucosidase, glucoxidase, peptizyme, and papain) and a metal ion management formulation to an oral cavity, maintaining the composition in the oral cavity for an extended period of time (e.g., about 30 seconds to about one minute), spitting out or removing the composition from the oral cavity, and repeating the process 2-3 times per day or as needed. In accordance with various aspects of these embodiments, the method of using the composition includes treatment of dry mouth, treatment or prevention of bad breath, treatment or prevention of a common cold, or the like.

In accordance with additional embodiments of the invention, a method of using the composition includes applying a foaming liquid including a plurality of enzymes selected from the group consisting of: lysozyme, amylase, amyloglucosidase, glucosidase, peptizyme, and papain (e.g., the composition may include lysozyme, amylase, amyloglucosidase, glucoxidase, peptizyme, and papain) and a metal ion management formulation to an interior portion of an oral cavity, such as on a tongue. In accordance with various aspects of these embodiments, the composition may be configured to foam during and/or after application to the oral cavity. In accordance with further aspects, the composition may be used to treat or prevent bad breath, to treat or prevent dry mouth symptoms, to treat a common cold, or the like.

In accordance with additional embodiments of the invention, a method of using the composition includes applying a liquid drop formulation (e.g., about 2-3 drops, each drop about 0.025 ml) including a plurality of enzymes selected from the group consisting of: lysozyme, amylase, amyloglucosidase, glucosidase, peptizyme, and papain (e.g., the composition may include lysozyme, amylase, amyloglucosidase, glucoxidase, peptizyme, and papain) and a metal ion management formulation to an interior portion of an oral cavity, such as on a tongue. In accordance with further aspects, the composition may be used to treat or prevent bad breath, to treat or prevent dry mouth symptoms, to treat a common cold, or the like.

In accordance with yet additional embodiments of the invention, a method of forming the composition includes the steps of mixing a solvent and a thickener (e.g., propanediol and carboxymethyl cellulose); adding a sweetener (e.g., xylitol), a healing agent or moisturizer (e.g., aloe vera), an active (e.g., zinc gluconate) and a surfactant (e.g., poloxamer 407) to another solvent (e.g., purified water) and mixing until the surfactant (e.g., poloxamer 407) is completely dissolved; adding the solvent-thickener mixture to the sweetener mixture; adding one or more pH stabilizers and/or preservatives (e.g., benzoic acid and/or sodium phosphate) slowly to the batch and mixing for about 15 minutes; measuring the pH; adding another pH stabilizer and an antibacterial agent (e.g., phytic acid and lactoferrin) to the batch and mixing for about 15 minutes; adding one or more enzymes selected from the group of lysozyme, glucoxidase, peptizyme, amylase, papain, and amyloglucosidase to the batch and mixing for about 15 minutes; and adding the natural flavor to the batch and mixing for about 30 minutes.

In accordance with yet additional embodiments of the invention, a method of forming the composition includes the steps of providing a solvent such as water, mixing in optional additional actives, a preservative, and moisturizer (e.g., aloe vera), mixing in a thickener/emulsifying agent, mixing in a surfactant, mixing in pH stabilizers, measuring a pH of the mixture, adding enzymes to the mixture, adding a sweetener to the mixture, adding any additional solvents to the mixture, and adding any flavoring agents to the mixture.

### DETAILED DESCRIPTION OF EXEMPLARY EMBODIMENTS

The present invention provides an improved oral composition and methods of forming and using the composition. As set forth in greater detail below, the oral composition, in accordance with various embodiments of the invention, includes enzymes to selectively reduce unwanted bacteria, selectively inhibit the growth of unwanted bacteria and/or reduce an amount of biofilm present in an oral cavity. The composition also facilitates or increases the moisture retention capabilities of oral mucous and reduces irritation in the oral cavity. The composition can be used for treatment and/or prevention of a variety of conditions, such as dry mouth, common cold, bad breath, or the like and can be used in the treatment of various animals, and is particularly well suited for the treatment of humans.

In accordance with various embodiments of the invention, the oral composition may be used to treat and/or prevent a variety of conditions, including bad breath, dry mouth, mouth sores and ulcers, plaque, tartar, gingivitis, gum disease, conditions leading to fluid overload, common colds, and other conditions.

By way of one particular example, the composition can be used to treat dry mouth in patients with chronic kidney disease to mitigate or prevent the onset of fluid overload. If left untreated, fluid overload may lead to heart failure and even death due to ventricular dystrophy. Indeed, fluid overload is thought to contribute to a significant number of deaths in chronic kidney disease patients due to ventricular dystrophy.

In accordance with various embodiments of the invention, a composition includes a plurality of enzymes that prevent formation of and/or break up of biofilm in an oral cavity and a metal ion management formulation.

In accordance with various embodiments of the invention, two or more of the plurality of enzymes are selected from the group consisting of: lysozyme, amylase, amyloglucosidase, glucoxidase, peptizyme, and papain. In accordance with a particular aspect, the composition includes lysozyme, amylase, amyloglucosidase, glucoxidase, peptizyme, and papain.

Lysozyme is a naturally occurring protein that attacks cell walls of certain bacteria (e.g., Clostridia bacteria). Lysozyme gradually weakens the walls of certain cells, causing the cells to eventually burst.

Amylase and amyloglucosidase are dextrinizing enzymes, which can break down complex foods containing sugars. Sugar in the mouth may lead to cavities, so breaking down the foods containing sugars helps reduce cavities and growth of unwanted bacteria.

Glucoxidase is a naturally occurring microbial enzyme that reacts with available oxygen to deprive aerobic bacteria of oxygen and thereby interfere with the aerobic bacteria growth.

Peptizyme is an anti-inflammatory enzyme that reduces dental infections and can help reduce an amount of plaque on a surface within an oral cavity.

Papain is a protease that helps reduce plaque on a surface of an oral cavity and also helps whiten teeth.

In accordance with various embodiments, the metal ion management formulation comprises one or more of: phytic acid, lactoferrin, and sodium EDTA. The metal ion management formulation is thought to be a basis for iron and other essential metal ion deprivation, which selectively inhibits growth of gram negative (“bad”) bacteria.

Lactoferrin is an iron binding glycoprotein, which is thought to react with iron required for pathogenic bacteria growth, destroying bacteria through iron deprivation. Phytic acid and sodium EDTA are chelating agents which bind to iron, to thereby inactivate the iron.

The composition of the present invention also includes a suitable carrier and may include other actives. In accordance with exemplary embodiments of the invention, the carrier includes a solvent such as purified water, one or more sweeteners and/or sugar alcohols, such as sorbitol and xylitol, preservatives, such as propanediol (propylene glycol) and sodium phosphate, a moisturizer or natural healing agent, such as aloe vera, thickeners or emulsifiers, such as carboxymethylcellulose, cellulose gum, polyvinyl pyrolidone, or hydroxylethyl cellulose, pH stabilizers, such as sodium phosphate and benzoic acid, a surfactant, such as Poloxamer 407, a preservative such as sodium benzoate, and natural flavorants, such as natural, mild mint flavoring and/or menthol.

The additional actives may include any active ingredients that facilitate oral health, such as actives configured to reduce plaque and/or provide a source of calcium. By way of example, zinc gluconate and/or calcium lactate may be an additional active ingredient.

Compositions in accordance with various embodiments of the invention exhibited the surprising an unexpected results of treating various conditions, as set forth below, without exhibiting metallic or other bad tastes typically associated with other oral rinse compositions.

### SPECIFIC EXAMPLES

The following non-limiting examples illustrate exemplary compositions in accordance with various embodiments of the invention. These examples are merely illustrative, and it is not intended that the invention be limited to the examples. Compositions in accordance with the present invention may include the ingredients listed below as well as additional and/or alternative inert materials, preservatives, and other constituents typically found in oral compositions. In the case where exemplary inert materials, preservatives, and/or the like are listed, these ingredients are merely exemplary, and it is understood that other similar ingredients may be substituted for the materials listed in the examples below.

### Example 1

A translucent oral composition, including the ingredients listed below, is formed by premixing the propanediol and carboxymethyl cellulose; adding xylitol, sorbitol, aloe vera, zinc gluconate and poloxamer 407 to purified water and mixing until the poloxamer 407 is completely dissolved; adding the propanediol/carboxymethyl mixture to the xylitol mixture; adding benzoic acid and sodium phosphate slowly to the batch and mixing for about 15 minutes or more; measuring the pH; adding phytic acid and lactoferrin to the batch and mixing for about 15 minutes; adding lysozyme, glucoxidase, peptizyme, amylase, papain, and amyloglucosidase to the batch and mixing for about 15 minutes; and adding the natural flavor to the batch and mixing for about 30 minutes.

The oral composition has a pH between about 5 and about 5.5 and has a specific gravity of about 1.01 to about 1.04.

| | | v/v | Exemplary Range |
|---|---|---|---|
| INGREDIENT | Function | ( % ) | v/v % |
| PURIFIED | SOLVENT | 59.6140 | 53.6526-65.575 |
| WATER | | | |
| XYLITOL | SWEETENER | 12.0000 | 10.8-13.2 |
| PROPANE | SOLVENT/ | 9.0000 | 8-9.9 |
| DIOL | PRESERVATIVE | | |
| SORBITOL | SOLVENT | 13.0000 | 11.7-14.3 |
| ALOE VERA | NATURAL | 0.2000 | 0.18-0.22 |
| | HEALING | | |
| | AGENT | | |
| SODIUM | PRESERVATIVE | 0.0010 | 0.0009-0.0011 |
| PHOSPHATE | | | |
| CARBOXY | EMULSIFIER | 5.0000 | 4.5-5.5 |
| METHYL | | | |
| CELLULOSE | | | |
| ZINC | FRESHENS | 0.0020 | 0.0018-0.0022 |
| GLUCONATE | BREATH | | |
| PHYTIC ACID | pH STABILISER | 0.0019 | 0.00171-0.00209 |
| BENZOIC ACID | pH STABILISER | 0.2500 | 0.225-0.00209 |
| POLOXAMER | SURFACTANT | 0.7500 | 0.675-0.825 |
| 407 | | | |
| LACTOFERRIN | ANTIBACTERIAL | 0.1000 | 0.09-0.11 |
| LYSOZYME | ANTIBACTERIAL | 0.0145 | 0.01305-0.01595 |
| GLUCOXIDASE | ANTIBACTERIAL | 0.0013 | 0.001188-0.001452 |
| PEPTIZYME | ANTIBACTERIAL | 0.0013 | 0.001134-0.001386 |
| AMYLASE | ANTIBACTERIAL | 0.0117 | 0.01053-0.01287 |
| PAPAIN | WHITENING | 0.0020 | 0.0018-0.0022 |
| AMYLO- | ANTIBACTERIAL | 0.0100 | 0.009-0.011 |
| GLUCOSIDASE | | | |
| NATURAL | FLAVORING | 0.0400 | 0.036-0.044 |
| FLAVOR | AGENT | | |
| TOTAL | | 100.000% | |

### Example 2

The composition of example 1 is used to treat dry mouth symptoms in a patient with chronic kidney disease. The patient is instructed to apply the composition of example 1 to an oral cavity, swish thoroughly for about 30 seconds to about one minute, and then spit out (discharge) the composition, and to repeat this application 2-3 times per day. The patient is further instructed to not immediately rinse the oral cavity after application of the composition or to eat food right after application of the composition. The method of example 2 reduced dry mouth symptoms, which, in turn, is thought to reduce or prevent fluid overload, which can lead to heart disease and even death due to ventricular dystrophy in patients suffering from chronic kidney disease.

### Example 3

The composition of example 1 is provided to a patient with diabetes that suffers from dry mouth, which results in cracked lips and mouth sores. The patient is instructed to apply the composition of example 1 to an oral cavity, swish thoroughly for about 30 seconds to about one minute, then spit out the composition, and to repeat this application 2-3 times per day. The patient is further instructed to not immediately rinse the oral cavity or eat food. The patient experienced relief from dry mouth and has not experienced any mouth sores while using the composition of example 1.

### Example 4

Another patient used the composition of example 1 according to the instructions of examples 2 and 3 and noticed a reduction of dry mouth and a corresponding reduction in a choking feeling during a period of using the composition of example 1 in accordance with the instructions provided.

### Example 5

A patient suffering from canker sores applied the composition of example 1 in accordance with the instructions of examples 2 and 3 and noticed a moisturizing effect of the composition. The patient further noticed that, unlike other purported remedies, the composition of example 1 did not sting upon application. The patient has not had a canker sore for three months while using the composition of example 1.

### Example 6

Another patient suffering from dry mouth and a sore roof of mouth used the composition of example 1 in accordance with the instructions of examples 2 and 3. The patient experienced relief from dry mouth, particularly upon waking, and elimination of soreness of the roof of the mouth.

### Example 7

Another patient used the composition of example 1 in accordance with the instructions of examples 2 and 3. The patient experienced improved breath (reduction in bad breath) and was able to cut down on fluid intake as a result of using the composition. The patient also noticed that her teeth felt better.

### Example 8

Another patient used the composition of example 1 in accordance with the instructions of examples 2 and 3. The patient did not experience dry mouth while using the composition. The patient also noticed a clean, wet, refreshed mouth in the morning and that her mouth generally felt clean as a result of using the composition.

### Example 9

A patient used the composition of example 1 in accordance with the instructions of examples 2 and 3. The patient is a mouth breather at night and consequently typically wakes up with dry mouth. The patient noticed that use of the composition helped maintain a moist mouth.

### Example 10

A patient suffering from chronic dry throat, itchy throat, and constant cough used the composition of example 1 in accordance with the instructions of examples 2 and 3. Surprisingly, the patient noticed that use of the composition helped maintain a moist throat and reduced or eliminated the itchy throat and cough, within one week. The patient continued to use the composition and did not experience a dry throat, itchy throat, or constant cough while continuing to use the composition.

### Example 11

A patient using the composition of example 1 for the treatment of dry mouth noticed an unexpected result that the composition did not have a metallic taste.

### Example 12

A patient using the composition of example 1 for the treatment of dry mouth noticed an unexpected result that a duration and a severity of his cold was reduced as a result of using the composition of example 1. The patient gargled with the composition 5-7 times and noticed a reduction in the severity of his cold within 12 hours.

### Example 13

A translucent oral composition, including the ingredients listed below, is formed by providing water to a mixing tank, mixing in the sodium benzoate, zinc gluconate, calcium lactate, and aloe vera and mixing for about 30 minutes or more, mixing in the thickener/emulsifier and mixing for about 45 minutes or until thickener/emulsifier dissolves, adding the surfactant and mixing for about 60 minutes or until surfactant is dissolved, adding the benzoic acid and disodium phosphate to the mixture and mixing for about 15 minutes or more, measuring the pH of the solution, adding enzymes and metal ion management compounds to the solution, mixing in the xylitol and mixing for about 30 minutes, adding the sorbitol and propylene glycol to the mixture and mixing for about 15 minutes, adding flavor agents to the mixture and mixing for about 15 minutes, and letting the mixture settle for about 15 minutes.

The oral composition has a pH between about 5.0 and 6.0 and has a specific gravity of about 1.02 to about 1.03.

| | | FORMULA | Exemplary Range |
|---|---|---|---|
| INGREDIENT | USE | (v/v % ) | (v/v %) |
| PURIFIED | SOLVENT | 63.2010% | 56.8809-69.5211 |
| WATER | | | |
| XYLITOL | SWEETENER | 12.0000% | 10.8-13.2 |
| PROPYLENE | SOLVENT/ | 9.0000% | 8.1-9.9 |
| GLYCOL | PRESERVATIVE | | |
| SORBITOL | SOLVENT | 13.0000% | 11.7-14.3 |
| ALOE VERA | NATURAL | 0.0100% | 0.009-0.011 |
| | HEALING | | |
| | AGENT | | |
| SODIUM | PRESERVATIVE | 0.3000% | 0.27-0.33 |
| BENZOATE | | | |
| POLYVINYL | | | |
| PYROLIDONE | EMULSIFIER | 0.5000% | 0.45-0.55 |
| ZINC | FRESHN'S | 0.0100% | 0.009-0.011 |
| GLUCONATE | BREATH | | |
| SODIUM | pH STABILISER | 0.0010% | 0.0009-0.0011 |
| PHOSPHATE | | | |
| CALCIUM | SOURCE FOR | 0.0200% | 0.018-0.022 |
| LACTATE | CALCIUM | | |
| BENZOIC ACID | pH STABILISER | 0.2500% | 0.225-0.275 |
| POLOXAMER | SURFACTANT | 1.5000% | 1.35-1.65 |
| 407 | | | |
| LACTOFERRIN | ANTIBACTERIAL | 0.0012% | 0.00108-0.00132 |
| LYSOZYME | ANTIBACTERIAL | 0.0015% | 0.001305-0.001595 |
| GLUCOXIDASE | ANTIBACTERIAL | 0.0013% | 0.001188-0.001452 |
| AMYLASE | ANTIBACTERIAL | 0.0012% | 0.001053-0.001287 |
| AMYLO- | ANTIBACTERIAL | 0.0014% | 0.00126-0.00154 |
| GLUCOSIDASE | | | |
| PEPTIZYME | ANTIBACTERIAL | 0.0012% | 0.00108-0.00132 |
| PAPAIN | WHITENING | 0.1000% | 0.09-0.11 |
| MINT FLAVOR | FLAVORING | 0.1000% | 0.09-0.11 |
| | AGENT | | |
| TOTAL | | 100.000% | |

### Example 14

A translucent oral composition, including the ingredients listed below, is formed by providing water to a mixing tank, mixing in the sodium benzoate, zinc gluconate, calcium lactate, and aloe vera and mixing for about 30 minutes or more, mixing in the thickener/emulsifier and mixing for about 45 minutes or until thickener/emulsifier dissolves, adding the surfactant and mixing for about 60 minutes or until surfactant is dissolved, adding the benzoic acid and disodium phosphate to the mixture and mixing for about 15 minutes or more, measuring the pH of the solution, adding enzymes and metal ion management compounds to the solution, mixing in the xylitol and mixing for about 30 minutes, adding the sorbitol and propylene glycol to the mixture and mixing for about 15 minutes, adding flavor agents to the mixture and mixing for about 15 minutes, and letting the mixture settle for about 15 minutes.

The oral composition has a pH between about 5.0 and 6.0 and has a specific gravity of about 1.02 to about 1.03.

| | | FORMULA | Exemplary Range |
|---|---|---|---|
| INGREDIENT | USE | (wt % ) | (wt %) |
| PURIFIED | SOLVENT | 67.105% | 60.3945-73.8155 |
| WATER | | | |
| XYLITOL | SWEETENER | 10.0000% | 9-11 |
| PROPYLENE | SOLVENT/ | 8.850% | 7.965-9.735 |
| GLYCOL | PRESERVATIVE | | |
| SORBITOL | SOLVENT | 12.750% | 11.7-14.3 |
| ALOE VERA | NATURAL | 0.015 | 0.0135-0.0165 |
| | HEALING | | |
| | AGENT | | |
| SODIUM | PRESERVATIVE | 0.3000% | 0.27-0.33 |
| BENZOATE | | | |
| HYDROXY- | EMULSIFIER | 0.3000% | 0.27-0.33 |
| ETHYL | | | |
| CELLULOSE | | | |
| ZINC | FRESHN'S | 0.0200% | 0.018-0.022 |
| GLUCONATE | BREATH | | |
| SODIUM | pH STABILISER | 0.015% | 0.0135-0.0165 |
| PHOSPHATE | | | |
| CALCIUM | SOURCE FOR | 0.0200% | 0.018-0.022 |
| LACTATE | CALCIUM | | |
| BENZOIC ACID | pH STABILISER | 0.04500% | 0.0.0405-0.0495 |
| PLURONIC | SURFACTANT | 0.350% | 0.315-0.385 |
| F127 | | | |
| LACTOFERRIN | ANTIBACTERIAL | 0.0012% | 0.00108-0.00132 |
| LYSOZYME | ANTIBACTERIAL | 0.0015% | 0.001305-0.001595 |
| GLUCOXIDASE | ANTIBACTERIAL | 0.0013% | 0.001188-0.001452 |
| AMYLASE | ANTIBACTERIAL | 0.0012% | 0.001053-0.001287 |
| AMYLO- | ANTIBACTERIAL | 0.0014% | 0.00126-0.00154 |
| GLUCOSIDASE | | | |
| PEPTIZYME | ANTIBACTERIAL | 0.0012% | 0.00108-0.00132 |
| PAPAIN | WHITENING | 0.1000% | 0.09-0.11 |
| MINT FLAVOR | FLAVORING | 0.005% | 0.0045-0.022 |
| | AGENT | | |
| MENTHOL | FLAVORING | 0.035% | 0.0315-0.0385 |
| | AGENT | | |
| TOTAL | | 100.000% | |

### Example 15

The composition of example 13 is used to treat a patient with dry mouth. The patient applied the composition of example 13 to an oral cavity, swished thoroughly for about 30 seconds to about one minute, then spit out the composition, and repeated this application 2-3 times per day. The patient noticed relief from (mitigation of) dry mouth and irritation. The patient noticed a moist and a clean and refreshed feeling within the mouth. The patient also noticed that there was no burning sensation noticed with application of the composition.

### Example 16

A patient suffering from canker sores applied the composition of example 13 in accordance with the instructions of example 15 and noticed that the canker sore was gone within days and has not reappeared while using the composition.

### Example 17

Another patient used the composition of example 13 in accordance with the instructions of examples 15 and 16. The patient did not experience dry mouth while using the composition. The patient also noticed a reduction in throat dryness/itching within one week of using the composition.

### Example 18

A translucent oral composition, including the ingredients listed below, is formed by premixing the propanediol, carboxymethyl cellulose, and xanthum gum; adding xylitol, aloe vera, zinc gluconate and poloxamer 407 to purified water and mixing until the poloxamer 407 is completely dissolved; adding the propanediol/carboxymethyl mixture to the xylitol mixture; adding benzoic acid and sodium phosphate slowly to the batch and mixing for about 15 minutes or more; measuring the pH; adding phytic acid and lactoferrin to the batch and mixing for about 15 minutes; adding lysozyme, glucoxidase, peptizyme, amylase, papain, and amyloglucosidase to the batch and mixing for about 15 minutes; and adding the natural flavor to the batch and mixing for about 30 minutes.

The oral composition has a pH between about 5 and about 5.5 and has a specific gravity of about 1.01 to about 1.04.

| | | FORMULA | Exemplary Range |
|---|---|---|---|
| INGREDIENT | USE | (w/v % ) | (w/v %) |
| PURIFIED | SOLVENT | 60.60% | 56.8809-69.5211 |
| WATER | | | |
| XYLITOL | SWEETNER | 12.00% | 10.8-13.2 |
| PROPANE | SOLVENT/ | 9.00% | 8.1-9.9 |
| DIOL | PRESERVATIVE | | |
| SORBITOL | SOLVENT | 13.00% | 11.7-14.3 |
| ALOE VERA | NATURAL | 0.01% | 0.009-0.011 |
| | HEALING | | |
| | AGENT | | |
| CARBOXY | EMULSIFIER | 0.50% | 0.20-1.5 |
| METHYL | | | |
| CELLULOSE | | | |
| ZINC | FRESHN'S | 0.01% | 0.009-0.011 |
| GLUCONATE | BREATH | | |
| SODIUM | Ph STABILISER | 0.001% | 0.0009-0.0011 |
| PHOSPHATE | | | |
| PHYTIC ACID | PRESERVATIVE | 0.01% | 0.01-0.1 |
| BENZOIC ACID | Ph STABILISER | 0.25% | 0.225-0.275 |
| POLOXAMER | SURFACTANT | 4.00% | 1.0-5.0 |
| 407 | | | |
| XANTHUM | THICKENER | 0.50% | 0.01-3.5 |
| GUM | | | |
| LACTOFERRIN | ANTIBACTERIAL | 0.001% | 0.00108-0.00132 |
| LYSOZYME | ANTIBACTERIAL | 0.0015% | 0.001305-0.001595 |
| GLUCOXIDASE | ANTIBACTERIAL | 0.0013% | 0.001188-0.001452 |
| AMYLASE | ANTIBACTERIAL | 0.0012% | 0.001053-0.001287 |
| AMYLO- | ANTIBACTERIAL | 0.0014% | 0.00126-0.00154 |
| GLUCOSIDASE | | | |
| PEPTIZYME | ANTIBACTERIAL | 0.0012% | 0.00108-0.00132 |
| PAPAIN | WHITENING | 0.10% | 0.09-0.11 |
| NATURAL | FLAVORING | 0.01% | 0.009-0.11 |
| FLAVOR | AGENT | | |
| TOTAL | | 100.00% | |

### Example 19

The composition of example 18 is used to treat a patient with dry mouth. The patient applied the composition of example 18 to an oral cavity, for about 30 seconds to about one minute, then spit out the composition, and repeated this application 2-3 times per day. The patient noticed relief from (mitigation of) dry mouth and irritation. The patient noticed a moist and a clean and refreshed feeling within the mouth. The patient also noticed that there was no burning sensation noticed with application of the composition.

### Example 20

The composition of example 18 was provided in liquid drop form. A patient applied 2-3 drops per application each drop about 0.025 ml. Similar results to examples 2-4, 6-12, 15, and 17 were observed in patients using the liquid drop formulation.

### Example 21

A patient using medical marijuana used the composition of example 18 and experienced relief from dry mouth symptoms.

### Example 22

A translucent oral foaming liquid composition, including the ingredients listed below, is formed by premixing the propanediol and carboxymethyl cellulose; adding xylitol and sorbitol, aloe vera, zinc gluconate and poloxamer 407 to purified water and mixing until the poloxamer 407 is completely dissolved; adding the propanediol/carboxymethyl mixture to the xylitol mixture; adding benzoic acid and sodium phosphate slowly to the batch and mixing for about 15 minutes or more; measuring the pH; adding phytic acid and lactoferrin to the batch and mixing for about 15 minutes; adding lysozyme, glucoxidase, peptizyme, amylase, papain, and amyloglucosidase to the batch and mixing for about 15 minutes; and adding the natural flavor to the batch and mixing for about 30 minutes.

The oral composition has a pH between about 5 and about 5.5 and has a specific gravity of about 1.01 to about 1.04.

| | | FORMULA | Exemplary Range |
|---|---|---|---|
| INGREDIENT | USE | (w/v % ) | (w/v %) |
| PURIFIED | SOLVENT | 61.10% | 56.8809-69.5211 |
| WATER | | | |
| XYLITOL | SWEETNER | 12.00% | 10.8-13.2 |
| PROPANE | SOLVENT/ | 9.00% | 8.1-9.9 |
| DIOL | PRESERVATIVE | | |
| SORBITOL | SOLVENT | 13.00% | 11.7-14.3 |
| ALOE VERA | NATURAL | 0.01% | 0.009-0.011 |
| | HEALING | | |
| | AGENT | | |
| CARBOXY | EMULSIFIER | 0.50% | 0.20-1.5 |
| METHYL | | | |
| CELLULOSE | | | |
| ZINC | BREATH | 0.01% | 0.009-0.011 |
| GLUCONATE | FRESHN'S | | |
| SODIUM | Ph STABILISER | 0.0010% | 0.0009-0.0011 |
| PHOSPHATE | | | |
| PHYTIC ACID | PRESERVATIVE | 0.01% | 0.01-0.1 |
| BENZOIC ACID | Ph STABILISER | 0.25% | 0.225-0.275 |
| POLOXAMER | SURFACTANT | 4.00% | 1.0-5.0 |
| 407 | | | |
| LACTOFERRIN | ANTIBACTERIAL | 0.0012% | 0.00108-0.00132 |
| LYSOZYME | ANTIBACTERIAL | 0.0015% | 0.001305-0.001595 |
| GLUCOXIDASE | ANTIBACTERIAL | 0.0013% | 0.001188-0.001452 |
| AMYLASE | ANTIBACTERIAL | 0.0012% | 0.001053-0.001287 |
| AMYLO- | ANTIBACTERIAL | 0.0014% | 0.00126-0.00154 |
| GLUCOSIDASE | | | |
| PEPTIZYME | ANTIBACTERIAL | 0.0012% | 0.00108-0.00132 |
| PAPAIN | WHITENING | 0.10% | 0.09-0.11 |
| NATURAL | FLAVORING | 0.01% | 0.009-0.11 |
| FLAVOR | AGENT | | |
| TOTAL | | 100.00% | |

### Example 23

The composition of example 22 is used to treat a patient with bad breath. The patient applied the composition of example 22 to surface (e.g., a tongue) of an oral cavity, for about 30 seconds to about one minute, then scraped the composition from the surface. The patient noticed relief from (mitigation of) bad breath.

Although exemplary embodiments of the present invention are set forth herein, it should be appreciated that the invention is not so limited. Various modifications, variations, and enhancements in the composition and method set forth herein may be made without departing from the spirit and scope of the present invention. Furthermore, the compositions set forth above may comprise, consist of, or consist essentially of the ingredients noted above.

What is claimed is:

1. An oral composition comprising:
a carrier comprising a solvent;
a plurality of enzymes selected from the group consisting of lysozyme, amylase, amyloglucosidase, glucosidase, peptizyme, and papain; and
a metal ion management formulation comprising one or more compounds selected from the group consisting of sodium EDTA, phytic acid, and lactoferrin.

2. The oral composition of claim 1, further comprising zinc gluconate.

3. The oral composition of claim 1, further comprising calcium lactate.

4. The oral composition of claim 1, wherein the solvent comprises purified water, propylene glycol, and sorbitol.

5. The oral composition of claim 1, wherein the carrier comprises a thickener selected from the group consisting of propanediol, carboxymethyl cellulose, cellulose gum, polyvinyl pyrolidone, hydroxylethyl cellulose, xanthum gum, and combinations thereof.

6. The oral composition of claim 1, wherein the carrier comprises a moisturizing agent.

7. A method of treating dry mouth symptoms using the oral composition of claim 1, the method comprising the steps of:
applying the composition of claim 1 to an oral cavity; and
discharging the composition.

8. An oral composition, the composition comprising:
a carrier comprising a solvent;
zinc gluconate;
a plurality of enzymes selected from the group consisting of lysozyme, amylase, amyloglucosidase, glucosidase, peptizyme, and papain; and
a metal ion management formulation comprising one or more compounds selected from the group consisting of sodium EDTA, phytic acid, and lactoferrin.

9. The oral composition of claim 8, wherein the pH of the composition is between about 5 and about 5.5.

10. The oral composition of claim 8, wherein the specific gravity of the composition is between about 1.01 and about 1.04.

11. The oral composition of claim 8, wherein the solvent comprises purified water, propylene glycol, and sorbitol.

12. The oral composition of claim 8, wherein the plurality of enzymes comprises lysozyme, amylase, amyloglucosidase, glucosidase, peptizyme, and papain.

13. The oral composition of claim 8, wherein the metal ion management formulation comprises phytic acid and lactoferrin.

14. The oral composition of claim 8 consisting essentially of:
one or more solvents;
a sweetener;
one or more preservatives;
a moisturizer;
an emulsifier;
the zinc gluconate;
one or more pH stabilizers;
the plurality of enzymes; and
the metal ion management formulation.

15. A method of using the oral composition of claim 8, the method comprising the steps of:
applying the composition of claim 8 to an oral cavity; and
discharging the composition.

16. A composition for treatment of dry mouth symptoms, the composition comprising:
a carrier comprising a solvent;
zinc gluconate;
calcium lactate;
a plurality of enzymes selected from the group consisting of lysozyme, amylase, amyloglucosidase, glucosidase, peptizyme, and papain; and
a metal ion management formulation comprising one or more compounds selected from the group consisting of sodium EDTA, phytic acid, and lactoferrin.

17. The composition for treatment of dry mouth symptoms of claim 16, wherein the plurality of enzymes comprises lysozyme, amylase, amyloglucosidase, glucosidase, peptizyme, and papain.

18. The composition for treatment of dry mouth symptoms of claim 16, wherein the metal ion management formulation comprises lactoferrin.

19. The composition for treatment of dry mouth symptoms of claim 16, further comprising a moisturizing agent.

20. A method of treating dry mouth symptoms using the composition for treatment of dry mouth symptoms of claim 16, the method comprising the steps of:
applying the composition of claim 16 to an oral cavity; and
discharging the composition.
